(19) United States
(12) Patent Application Publication
(10) Pub. No.: US 2014/0098049 A1
(43) Pub. Date: Apr. 10, 2014

(54) SYSTEMS AND METHODS FOR TOUCH-BASED INPUT ON ULTRASOUND DEVICES

(71) Applicant: FUJIFILM Sonosite, Inc., Bothell, WA (US)

(72) Inventors: Axel Koch, Renton, WA (US); Jason Fouts, Bothell, WA (US)

(73) Assignee: FUJIFILM SONOSITE, INC., Bothell, WA (US)

(21) Appl. No.: 14/049,182

(22) Filed: Oct. 8, 2013

Related U.S. Application Data
(60) Provisional application No. 61/711,185, filed on Oct. 8, 2012.

(51) Int. Cl. G06F 3/01; G06F 3/041; A61B 8/00

(52) U.S. Cl. CPC G06F 3/016; A61B 8/467; G06F 3/041
USPC 345/173

(57) ABSTRACT

Systems and methods for receiving touch-based input from an operator of an imaging device are disclosed herein. In one embodiment, an ultrasound imaging device is configured to receive tactile input from an operator. The imaging device presents an ultrasound image to the operator and the operator can perform one or more touch inputs on the image. Based on the received input, the imaging device can update the display of the image.

### CROSS-REFERENCE TO RELATED APPLICATION

The present application claims priority under 35 U.S.C. 119(e) to U.S. Provisional Application Ser. No. 61/711,185, filed Oct. 8, 2012, the disclosure of which is incorporated herein by reference in its entirety.

### TECHNICAL FIELD

The disclosed technology relates generally to touch-based user input and in particular to systems and methods for receiving touch-based user input on ultrasound imaging devices.

### BACKGROUND

In ultrasound imaging devices, images of a subject are created by transmitting one or more acoustic pulses into the body from a transducer. Reflected echo signals that are created in response to the pulses are detected by the same or a different transducer. The echo signals cause the transducer elements to produce electronic signals that are analyzed by the ultrasound system in order to create a map of some characteristic of the echo signals such as their amplitude, power, phase or frequency shift etc. The map therefore can be displayed to a user as images.

Many ultrasound imaging devices include a screen for displaying ultrasound images and a separate input device (e.g., a hardware control panel and/or keyboard) for inputting commands and adjusting the display of the images on the screen. Use of a control panel to adjust ultrasound images can be awkward and cumbersome, as an operator may have to manipulate several variables simultaneously to adjust the image to his or her liking. Furthermore, inputting commands using a control panel may require that the operator break visual contact with the image display to focus on the control panel. In addition, a control panel on an ultrasound imaging device may include several commands and/or functions, requiring an operator to undergo extensive training before becoming proficient in using the device. A need exists for an intuitive ultrasound image display system that reduces the need for an operator to break visual contact with the display while decreasing time spent adjusting images on the display.

### BRIEF DESCRIPTION OF THE DRAWINGS

FIGS. 1A and 1B are isometric front and rear views, respectively, of an ultrasound imaging system configured in accordance with an embodiment of the disclosed technology.

FIG. 2 is a block diagram showing the components of an ultrasound imaging system in accordance with an embodiment of the disclosed technology.

FIGS. 3A-3F illustrate suitable user interface methods for manipulation of an ultrasound image in accordance with an embodiment of the disclosed technology.

FIG. 4 is a flow diagram illustrating a process for receiving user input in accordance with an embodiment of the disclosed technology.

### DETAILED DESCRIPTION

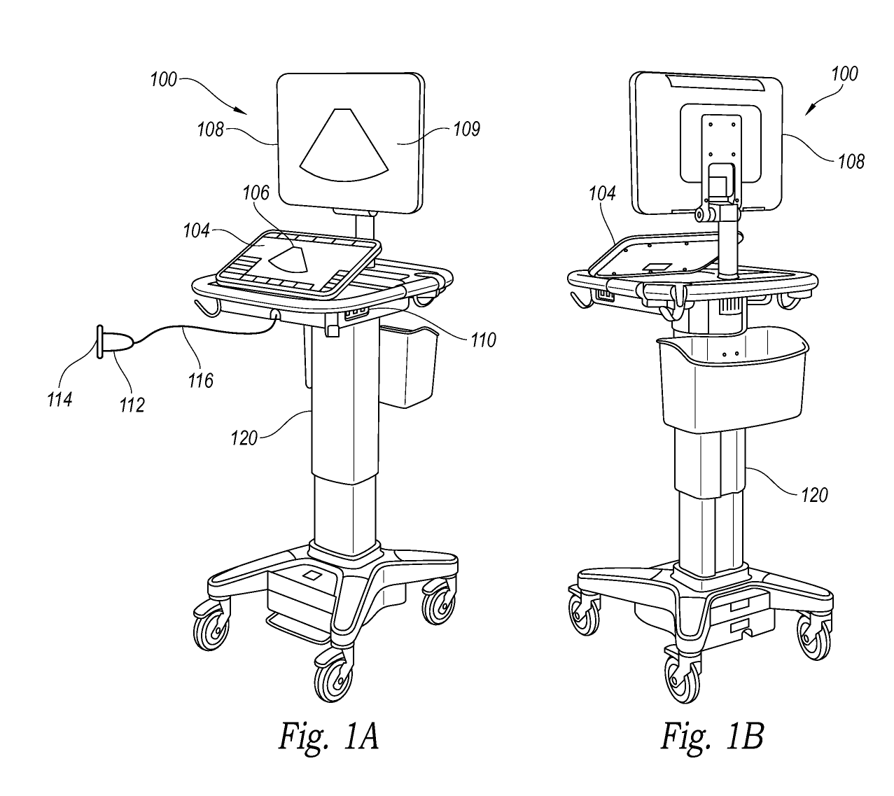

The present technology is generally directed to ultrasound imaging devices configured to receive touch-based input. It will be appreciated that several of the details set forth below are provided to describe the following embodiments in a manner sufficient to enable a person skilled in the relevant art to make and use the disclosed embodiments. Several of the details described below, however, may not be necessary to practice certain embodiments of the technology. Additionally, the technology can include other embodiments that are within the scope of the claims but are not described in detail with reference to FIGS. 1-4.

FIGS. 1A and 1B are front and rear isometric views, respectively, of an ultrasound imaging device 100 configured in accordance with an embodiment of the disclosed technology. In the illustrated embodiment, the device 100 includes a first display 104 (e.g., a touchscreen display) and a second display 108, each coupled (e.g., via a cable, wirelessly, etc.) to a processing unit 110. The first display 104 is configured to present a first display output 106 (e.g., a user interface and/or ultrasound images) to an operator of the ultrasound imaging device 100. Similarly, the second display 108 is configured to present a second display output 109 (e.g., a user interface and/or ultrasound images). A support structure 120 holds the device 100 and allows the operator to move the device 100 and adjust the height of the first and second displays 104 and 108.

The processing unit 110 can be configured to receive ultrasound data from a probe 112 having an ultrasound transducer array 114. The array 114 can include, for example, a plurality of ultrasound transducers (e.g., piezoelectric transducers) configured to transmit ultrasound energy into a subject and receive ultrasound energy from the subject. The received ultrasound energy may then be transmitted as one or more ultrasound data signals via a link 116 to the ultrasound processing unit 110. The processing unit 110 may be further configured to process the ultrasound signals and form an ultrasound image, which can be included in the first and second display outputs 106 and 109 shown on the displays 104 and 108, respectively.

In the example shown, either of the displays 104 and 108 may be configured as a touchscreen, and the processing unit 110 can be configured to adjust the display outputs 106 and 109, respectively based on touch-based input received from an operator. The displays 104 and 108 can include any suitable touch-sensitive display system such as, for example, resistive touchscreens, surface acoustic wave touchscreens, capacitive touchscreens, surface capacitance touchscreens, projected capacitance touchscreens, mutual capacitance touchscreens, self-capacitance touchscreens, infrared touchscreens, optical imaging touchscreens, dispersive signal touchscreens, acoustic pulse recognition touchscreens, etc. In addition, the displays 104 and 108 can be configured to receive input from a user via one or more fingers (e.g., a fingertip, a fingernail, etc.), a stylus, and/or any other suitable pointing implement.

In operation, for example, the operator may hold the probe 112 with a first hand while adjusting the ultrasound image presented in the display output 106 with a second hand, using, for example, one or more touch-based inputs or gestures. These inputs may include, for example, direct manipulation (e.g., dragging one or more fingers on the display 104 to move an element on the display output 106), single and double tapping the display 104 with one or more fingers, flicking the display 104 with one or more fingers, pressing and holding one or more fingers on the display 104, pinching and expanding two or more fingers on the display 104, rotating two or more fingers on the display 104, etc. As explained in further detail below, the processing unit 110 can be configured to receive the inputs from the display 104 and update the display output 106 to correspond the operator input.

As noted above, the display output 106 may include a user interface (UI) to control measurements and/or output of the device 100. In some embodiments, for example, the display output 109 may be similar or identical to the display output 106. In other embodiments, however, the display output 109 may be tailored for persons within close proximity to the device 100 (e.g., a patient and/or a physician). For example, the display output 109 may include larger sized renderings of ultrasound images formed by the processing unit 110 compared to those display in the display output 106. In other embodiments, either of the display outputs 106 and 109 can be configured for direct manipulation. For example, the display outputs 106 and 109 can be configured such that there is generally a one-to-one size relationship between a region in the subject being imaged and the image presented to the operator. This can offer the advantage of allowing the operator an intuitive experience when interacting with the image.

In illustrated embodiment of FIG. 1, the ultrasound imaging device 100 includes the two displays 104 and 108. In some embodiments, however, the device 100 may include additional displays or include only the display 104. In other embodiments, the displays 104 and 108 may be physically separated from the processing unit 110 and configured to wirelessly communicate with the processing unit 110 to, for example, transmit inputs received from an operator and/or receive the display outputs 106 and 109, respectively. Furthermore, in some embodiments, both of the displays 104 and 108 may be touch-sensitive, while in other embodiments, only the first display 104, for example, may be touch-sensitive.

In some other embodiments, the device 100 may comprise the display 104 and the processing unit 110 as a single integrated component. For example, the ultrasound imaging device 100 may comprise a handheld portable ultrasound system having the display 104, the processing unit 110, and the probe 112, without the support structure 120.

The technology disclosed herein allows an operator to collect ultrasound images of a subject while manipulating the images on a first display without looking away, for example, from the second display while operating the imaging device. The disclosed technology allows the operator to manipulate the image using an interface having intuitive touch-based inputs, reducing the time spent learning a set of commands associated with a hardware control panel. Furthermore, in some embodiments of the disclosed technology, the user interface is provided on a touchscreen display with a flat, cleanable surface, allowing the operator to more effectively disinfect the input area than many conventional prior art input devices.

### Suitable System

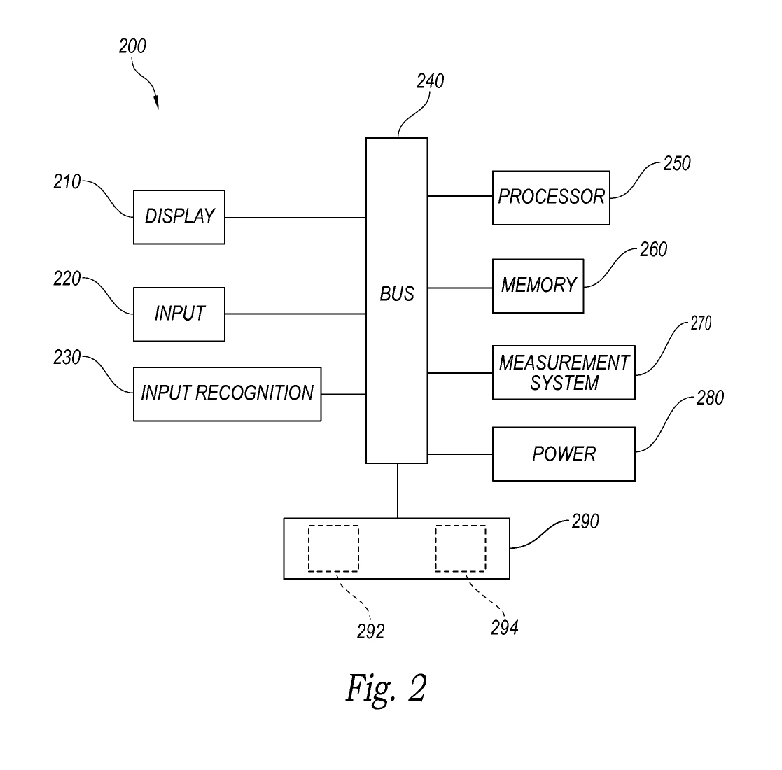

FIG. 2 and the following discussion provide a brief, general description of a suitable environment in which the technology may be implemented. Although not required, aspects of the technology are described in the general context of computer-executable instructions, such as routines executed by a general-purpose computer (e.g., an ultrasound imaging device processing unit). Aspects of the technology can be embodied in a special purpose computer or data processor that is specifically programmed, configured, or constructed to perform one or more of the computer-executable instructions explained in detail herein. Aspects of the technology can also be practiced in distributed computing environments where tasks or modules are performed by remote processing devices, which are linked through a communication network. In a distributed computing environment, program modules may be located in both local and remote memory storage devices.

Aspects of the technology may be stored or distributed on computer-readable media, including magnetically or optically readable computer disks, as microcode on semiconductor memory, nanotechnology memory, organic or optical memory, or other portable data storage media. Indeed, computer-implemented instructions, data structures, screen displays, and other data under aspects of the technology may be distributed over the Internet or over other networks (including wireless networks), on a propagated signal on a propagation medium (e.g., an electromagnetic wave(s), a sound wave, etc.) over a period of time, or may be provided on any analog or digital network (packet switched, circuit switched, or other scheme).

Referring to FIG. 2, a block diagram illustrating example components of an ultrasound imaging system 200 is shown. In the embodiment shown in FIG. 2, the system 200 includes a display 210, an input 220, an input recognition engine 230, a bus 240, one or more processors 250, memory 260, a measurement system 270, and power 280.

The display 210 can be configured to display, for example, a user interface to receive commands from an operator and/or present measured ultrasound images. The display 210 may include any suitable visual and/or audio display system such as, for example, a liquid crystal display (LCD) panel, a plasma-based display, a video projection display, etc. While only one display 210 is shown in FIG. 2, those of ordinary skill in the art would appreciate that multiple displays having similar or different outputs may be implemented in the system 200, as explained above with reference to FIGS. 1A and 1B.

In some embodiments, the input 220 may be implemented as a touch-sensitive surface on the display 210. In other embodiments, the input 220 may include additional inputs such as, for example, inputs from a control panel, a keyboard, a trackball, a system accelerometer and/or pressure sensors in the touch screen, audio inputs (e.g., voice input), visual inputs, etc. In further embodiments, the input 220 may be configured to receive non-tactile gestures performed by an operator without contacting a surface. In these embodiments, for example, the system 200 may include one or more sensors (e.g., one or more cameras, one or more infrared transmitters and/or receivers, one or more laser emitters and/or receivers, etc.) configured to detect, for example, one or more operator hand movements. The system 200 can be configured to analyze the operator hand movements and perform a corresponding action associated with the hand movements.

The system 200 can receive input from an operator at the input 220 (e.g., one or more touchscreen displays), which can be converted to one or more input signals and transmitted to the input recognition engine 230 and/or the processor 250. The input signals may include, for example, X-Y coordinate information of the tactile contact with the input 220, the time duration of each input, the amount of pressure applied during each input, or a combination thereof. The input recognition engine 230 can, based on the input signals, identify the input features (e.g., taps, swipes, dragging, etc.) and relay information regarding the identified input features to the one or more processors 250.

The processors 250 can perform one or more corresponding actions (e.g., adjusting an image output to the display 210) based on the identified input features from the input recognition engine 230. The input recognition engine 230, for example, can be configured to detect the presence of two of the operator's fingers at the input 220 in an area corresponding to the output of an ultrasound image on the display 210. For example, the operator may place his or her two fingers on an image and subsequently move them apart in a “pinch and expand” motion, which may be associated with zooming in on or expanding the view of an area of interest in the image display. The input recognition engine 230 can identify the pinch and expand input and the one or more processors 250 can correspondingly update the output to the display 210 (e.g., increase the zoom level of the currently displayed image at the region where the finger movement was detected).

The system 200 may control components and/or the flow or processing of information or data between components using one or more processors 250 in communication with the memory 260, such as ROM or RAM (and instructions or data contained therein) and the other components via a bus 260. The memory 260 may, for example, contain data structures or other files or applications that provide information related to the processing and formation of ultrasound images. The memory may also, for example, contain one or more instructions for providing an operating system and/or a user interface configured to display commands and receive input from the operator.

The measurement system 270 can be configured to transmit and receive ultrasound energy into a subject (e.g., a patient) and send acquired ultrasound data to the processor 250 for image processing. The measurement system 270 can include, for example, an ultrasound probe (e.g., the probe 112 in FIGS. 1A and 1B). For example, the measurement system 270 may include an array of transducers made from piezoelectric materials, CMUTs, PMUTs, etc. The measurement system 270 may also include other measurement components associated with a suitable ultrasound imaging modality such as, for example, a photoacoustic emission system, a hemodynamic monitoring system, respiration monitoring, ECG monitoring, etc.

Components of the system 200 may receive energy via a power component 280. Additionally, the system 200 may receive or transmit information or data to other modules, remote computing devices, and so on via a communication component 235. The communication component 235 may be any wired or wireless components capable of communicating data to and from the system 200. Examples include a wireless radio frequency transmitter, infrared transmitter, or hard-wired cable, such as a USB cable. The system 200 may include other additional component 290 having modules 292 and 294 not explicitly described herein, such as additional microprocessor components, removable memory components (flash memory components, smart cards, hard drives), and/or other components.

### User Interface

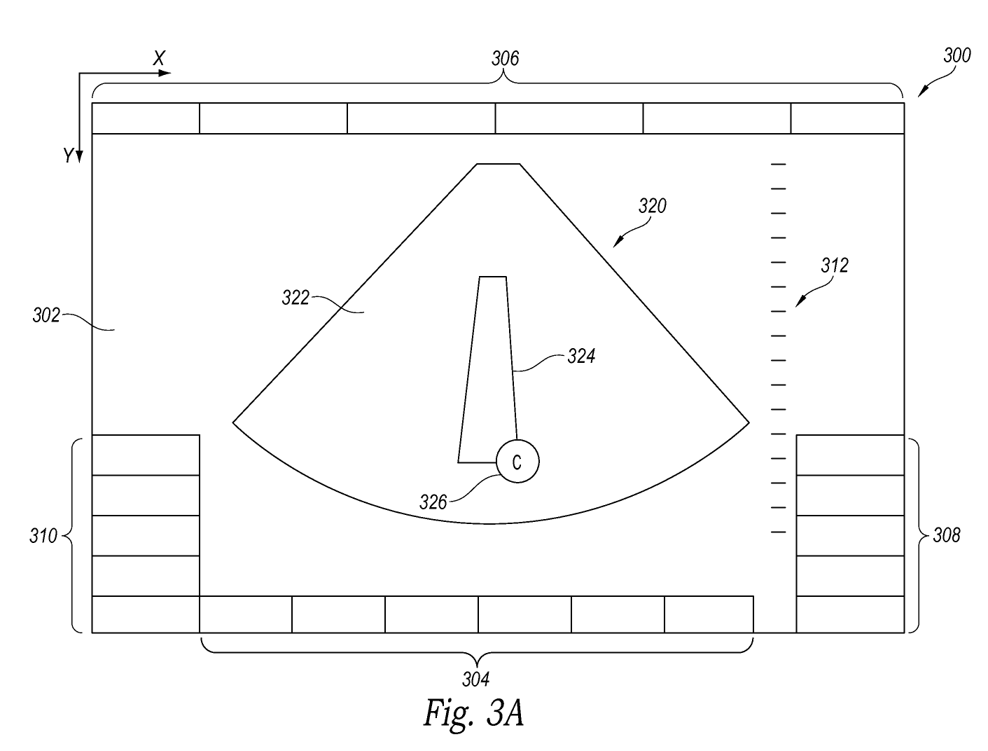

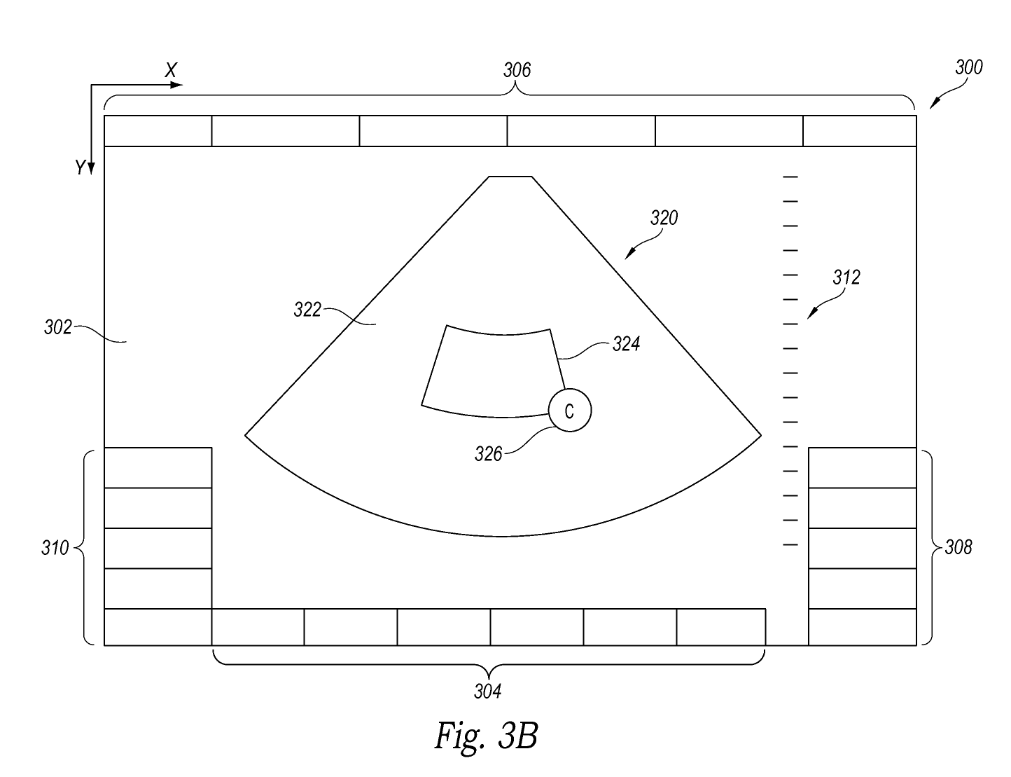

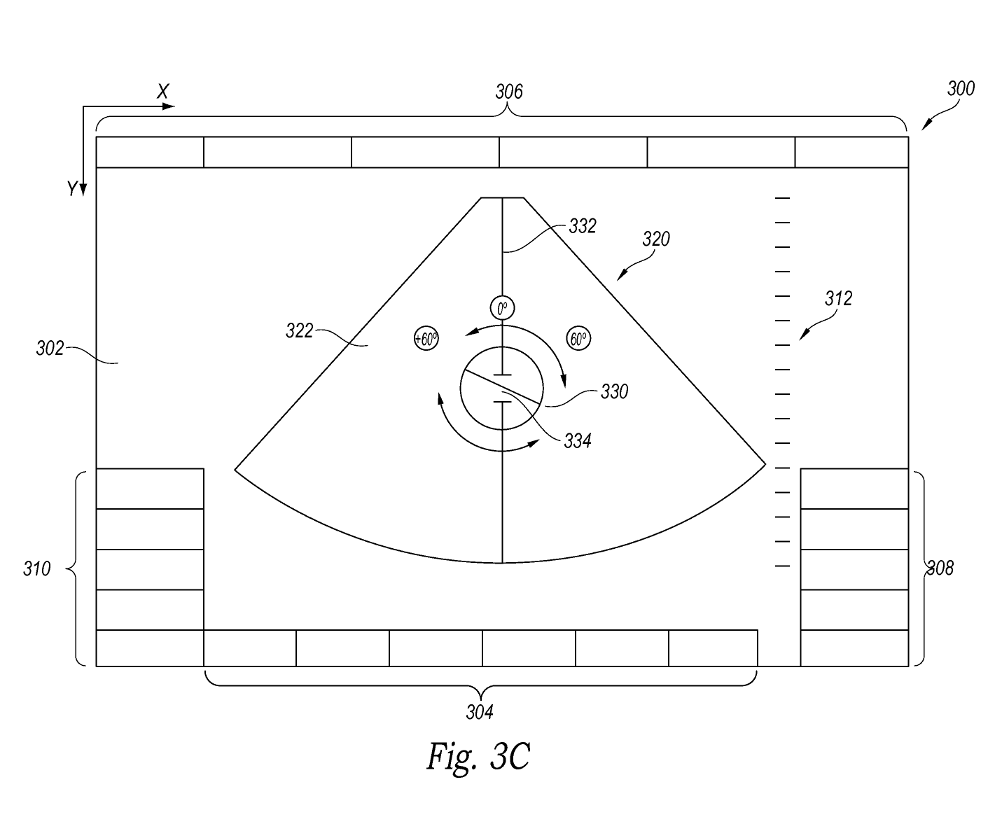

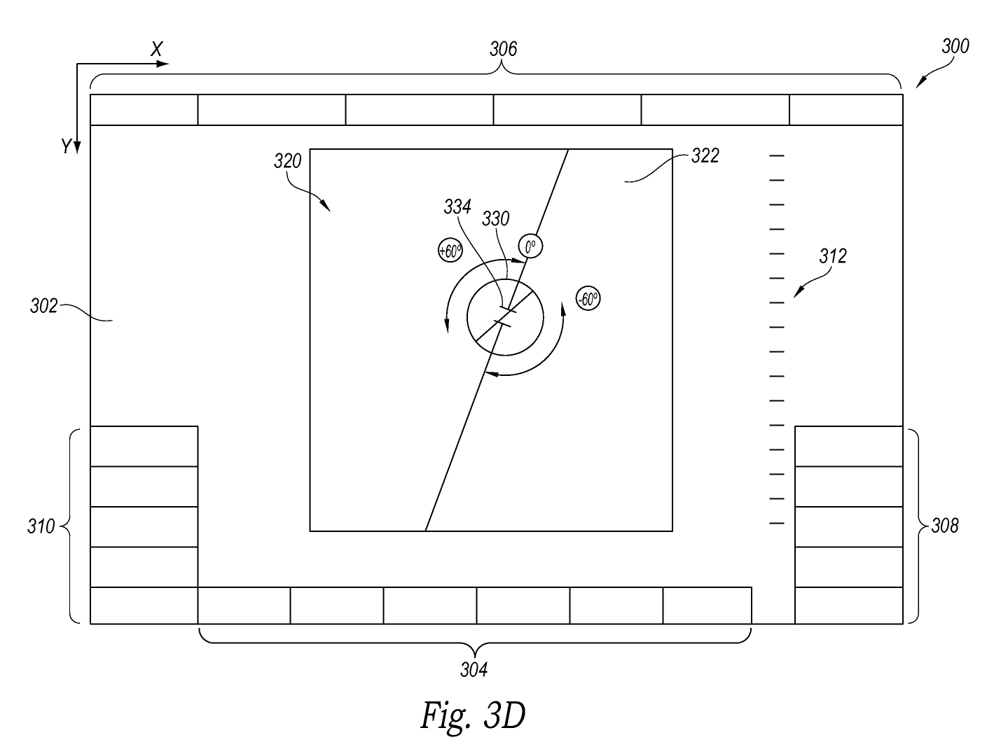

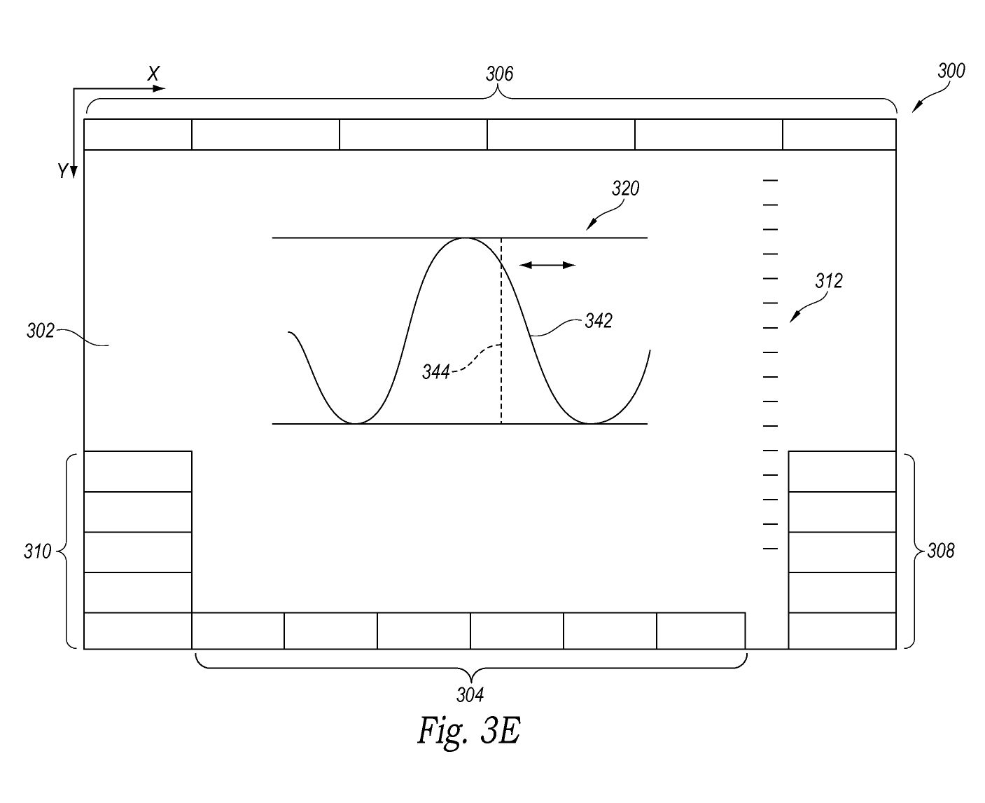

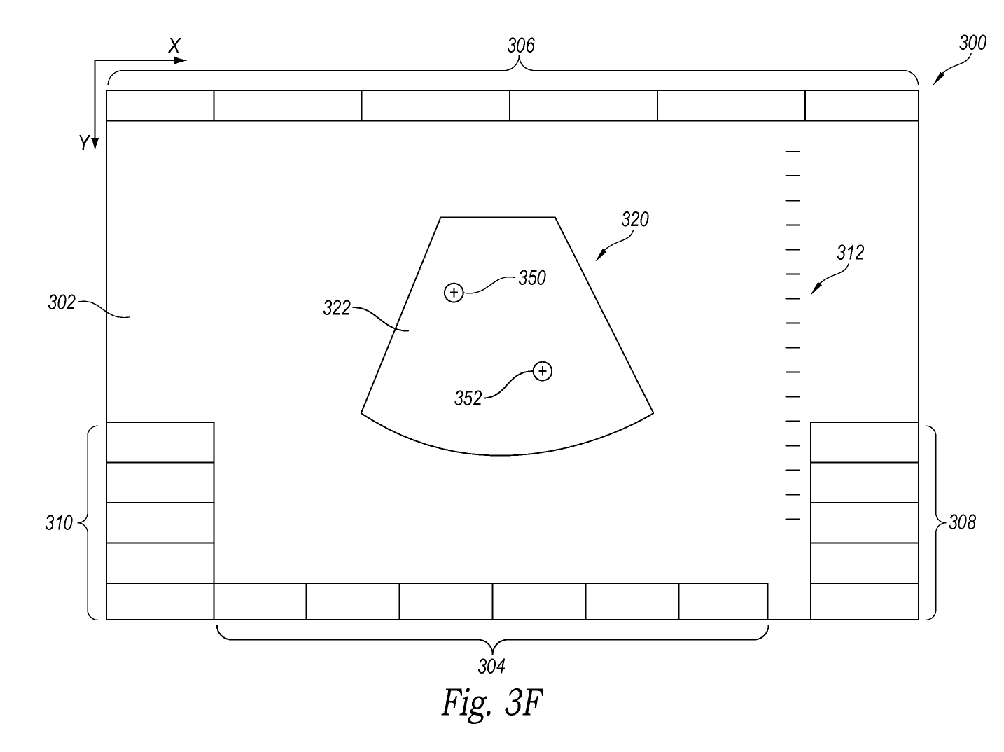

FIG. 3A illustrates a display 300 that includes a user interface 302 suitable for manipulating an ultrasound image and/or controlling an acquisition of one or more ultrasound images in response to receiving operator inputs (e.g., a mixture of stroke or traces, as well as taps, hovers, and/or other tactile inputs using one or more fingers). In the example shown in FIG. 3A, the user interface 302 is configured for use on an ultrasound device (e.g., the ultrasound imaging device 100 in FIGS. 1A and 1B) and presented to an operator. As those skilled in the art would appreciate, however, the user interface described herein may form part of any system where it is desirable to receive operator tactile input and perform one or more associated actions. Such systems may include, for example, mobile phones, personal display devices (e.g., electronic tablets and/or personal digital assistants), portable audio devices, portable and/or desktop computers, etc.

The user interface 302 is configured to present output and input components to an operator. A first user control bar 304, a second user control bar 306, a third user control bar 308, and a fourth user control bar 310 present icons to the operator associated with, for example, various operating system commands (e.g., displaying an image, saving an image, etc.) and/or ultrasound measurements. A adjustable scale 312 can be configured to adjust image generation, measurement, and/or image display parameters such as, for example, time, dynamic range, frequency, vertical depth, distance, Doppler velocity, etc.

Referring to FIGS. 3A and 3B, an ultrasound image display region 320 displays one or more ultrasound images 322 formed from, for example, ultrasound data acquired by an ultrasound measurement system (e.g., the measurement system 270 described above in reference to FIG. 2). A color box or region of interest (ROI) boundary 324 can be adjusted by the operator to select a particular area in the image 322. For example, the operator can adjust a shape (e.g., a square, rectangular, trapezoid, etc.) or a size of the boundary 324 to include a ROI in the image 322. The operator can also move the boundary 324 horizontally (e.g., along the x-axis) or vertically (e.g., along the y-axis) within the display region 320 to select the portion of the image he or she is interested in viewing.

In the illustrated examples shown in FIG. 3A and 3B, the interface 302 can be configured to receive operator tactile input in order to adjust the shape and size of the boundary 324 in an efficient, fast, and intuitive manner without breaking visual contact with the image 322. For example, an operator can touch the display region 320 shown in FIG. 3A with two fingers (e.g., a thumb and an index finger). The operator, while keeping the two fingers in contact with the display 300, can subsequently move the fingers apart on the display 300 until a desired shape and/or size of the boundary 324 is displayed within the image 322. The operator can further adjust the boundary 324, for example, by touching and holding a contact point 326 in and/or on the boundary 324 to re-size and/or reposition the boundary 324 to a desired size and shape.

In some embodiments, for example, the boundary 324 can also be adjusted through the use of other touch-based input and/or gestures. For example, the interface 302 may be configured to recognize a double tap input (e.g. multiple touch based input by one or more fingers in the same general location) and correspondingly display an expanded view (e.g., zoomed view) of the image within the boundary 324. In other embodiments, for example, the boundary 324 may be configured to allow the operator to resize the boundary 324 in only one dimension. For example, the boundary 324 can be configured to allow adjustment in only the horizontal (x) or only the vertical (y) dimension, as opposed to conventional “pinch and expand” gestures, which may simply scale a user interface element at the same rate in both directions (i.e. the scaling only depends on the distance between the two contact points).

In further embodiments, the user interface 302 can be configured to receive a gesture from the operator associated with, for example, a freeze command to freeze the current image (e.g., the image 322) displayed in the display region 320. For example, the user interface 302 may be configured to associate a gesture with a freeze command. In conventional ultrasound display systems, the operator may have to break visual contact with an ultrasound image (e.g. the image 322) to find a freeze button on a control panel. The present system, however, allows the operator to use the gesture anywhere in and/or on the user interface 302 without breaking visual contact with the display. For example, the user interface can be configured to receive a two-finger double-tap from the operator and accordingly freeze the image 322. A two-finger double-tap can offer the advantage of avoiding false positives that may occur with, for example, a single-finger gesture (e.g., an operator's accidentally freezing the image when he or she intended to do something totally different, like pressing a button).

FIGS. 3C and 3D illustrate the user interface 302 with the display region 320 in a Doppler display mode. In the illustrated embodiment of FIG. 3C, the image 322 is a Doppler image and a Doppler line 332 and a Doppler gate 334 are shown within the display region 320. The user interface 302 can be configured to receive operator touch-based input to adjust the size of the Doppler gate 334. For example, the operator can place two or more fingers within the display region 320 and move them toward or away from each other to contract or expand, respectively, the size of the Doppler gate 334.

Referring to FIGS. 3C and 3D, the user interface 302 can also be configured to receive rotational touch-based input from an operator to, for example, control a steering angle display 330 of the Doppler gate 334. For example, the operator can place one or more fingers on the display 300 at the steering angle display 330 and rotate the fingers in contact with the display 300 relative to each other. The ultrasound system (e.g. the system 200 described above in reference to FIG. 2) can be configured to rotate the Doppler gate measurement accordingly to adjust the angle control of the Doppler gate while the user interface 302 updates the Doppler gate 334 in the display region 320, as shown in FIG. 3D.

In the examples shown in FIGS. 3C and 3D, the user interface 302 and be configured to allow the operator to adjust the display of the image 322 with one or more of the following gestures.

Pinching and expanding the Doppler gate 330 (e.g., increasing or decreasing the distance between the two contact points will increase or decrease, respectively, the Doppler gate size). In some embodiments, for example, the x and y components of the movement may not considered, and only the pixel distance between the contact points may be taken into consideration.

A multi-touch rotational gesture may, for example, be associated with adjusting the angle correction display 330. For example, the operator may place two fingers (e.g., a finger and a thumb) on the angle correction display 330 or within the display region 320. While holding the two fingers approximately the same distance apart from each other, the operator may rotate the fingers in a circular pattern clockwise or counterclockwise to correspondingly adjust the angle correction display 330 (e.g., to adjust a Doppler angle). The operator can perform the rotational gesture until the Doppler gate 334 is suitably aligned with an area of interest in the image 322. While holding the fingers in the same position, the operator may also move the Doppler gate 332 to another location within the image 322. As those skilled in the art would appreciate, the operator may also use any other combination of fingers to perform the rotational gesture (e.g., an index finger and a middle finger, a first finger on a first hand and a second finger on a second hand, etc.). In some embodiments, the user interface 302 can be configured to receive a circular tactile input with which the operator can trace, for example, a rotational path of the angle correction display 330 with one or more fingers. In further embodiments, the user interface can be configured to receive three or more separate tactile inputs (e.g., three or more fingers) to rotate the angle correction display 330.

An accelerated single touch movement (e.g. a flick) within the display region 320 may be interpreted to control a steering angle of the Doppler line control for linear transducers. For example, the operator may apply the accelerated single touch movement to the Doppler line 332 to adjust an angle thereof to suitably align the Doppler gate 334 with the image 322.

An operator may also use, for example, a single touch and drag along the Doppler line 332 to correspondingly align the Doppler gate 334 along ultrasound ray boundaries (e.g., horizontally for linear transducers and at an angle for phased or curved transducers).

FIG. 3E illustrates the user interface 302 with the display region 320 in a wave form display mode. In the embodiment of FIG. 3D, the display region 320 includes, for example, an ECG waveform 342 and an ECG delay line 344 that can be manipulated by an operator using touch-based input. As those of ordinary skill in the art would appreciate, the ECG waveform 342 may be monitored by the operator to determine, for example, during which intervals ultrasound images and/or video clips should be acquired. The delay line 344 can be configured to indicate an operator-desired position on the ECG waveform 342 at which an ultrasound video clip acquisition is triggered. The operator can, for example, touch and hold the delay line 344 to adjust the horizontal position (e.g., along the x-axis) of the delay line 344 until a desired position of triggering along the ECG waveform 342 is reached. Additionally, for example, the user interface 302 can be configured to receive touch-based input to allow the operator to change the gain of the ECG waveform 342 and/or to pan or scroll along the ECG waveform 342 (e.g., using a flick, swipe, and/or dragging input) to view additional portions of the ECG waveform 342. As those of skill in the art would appreciate, the user interface 302 can be configured to display any suitable waveform to the operator such as, for example, a respiration waveform of a subject, Doppler trace data, M-Mode trace data, etc.

FIG. 3F illustrates the user interface 302 with the display region 320 in a caliper measurement mode. In the embodiment of FIG. 3F, the display region 320 includes the ultrasound image 322 and a first measurement point 350 and a second measurement point 352. As explained below, the measurement points 350 and 352 can be configured to be placed within an image. Measurement information associated with a portion of the ultrasound image 322 between the measurement points 350 and 352 can be calculated and presented to the user. In the illustrated embodiment, the two measurement points 350 and 352 are shown. In some embodiments, however, there may be several pairs of measurement points, each pair configured to be associated with a discrete measurement. In other embodiments, for example, the operator can trace one or more fingers on the display 300 within the display region 320 to indicate a desired measurement region (e.g., for the measurement of a diameter, area, and/or circumference of the measurement region). In further embodiments, measurement points may be placed individually within the ultrasound image 322 rather than as pairs.

In the example shown in FIG. 3F, the user interface 302 can be configured to receive, for example, tactile input from the operator to place the measurement points 350 and 352 at desired locations within the ultrasound image 322. Based on the locations of the measurement points 350 and 352, the system 200 (as described above with reference to FIG. 2) can calculate and display measurement information associated with a portion of the ultrasound image between the measurement points 350 and 352. For two-dimensional measurements, for example, the measurement information may include a distance between the two measurement points 350 and 352. For M-Mode measurements, for example, the measurement information may include a distance, a heart rate, and/or an elapsed time in the portion of the ultrasound image 322 between the measurement points 350 and 352. For Doppler measurements, for example, the measurement information may include a velocity, a pressure gradient, an elapsed time, a +/× ratio, a Resistive Index, an acceleration, etc. between the measurement points 350 and 352.

### Suitable Input Methods

The flow diagrams described herein do not show all functions or exchanges of data, but instead provide an understanding of commands and data exchanged under the system. Those skilled in the relevant art will recognize that some functions or exchange of commands and data may be repeated, varied, omitted, or supplemented, and other (less important) aspects not shown may be readily implemented. Further, although process steps, method steps, blocks, algorithms or the like may be described in a particular order, such processes, methods, blocks and algorithms may be configured to work in alternate orders. In other words, any sequence or order described herein does not necessarily indicate a requirement that the steps or blocks be performed in that order. The steps or blocks of processes and methods described herein may be performed in any order practical, and some steps may be performed simultaneously.

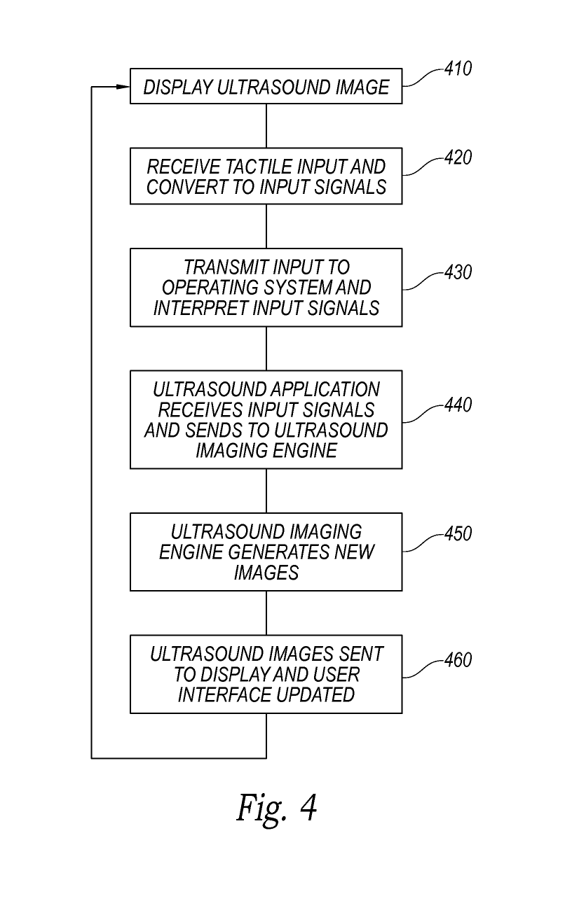

FIG. 4 shows a process 400 for receiving tactile input from an operator of an ultrasound imaging device. At block 410, an image (e.g., a two-dimensional, three-dimensional, M-Mode, and/or Doppler ultrasound image) is presented to an operator on a display (e.g., a touchscreen), while the process 400 monitors the display for operator input (e.g., detecting one or more fingers in contact with the display at the location of the image within the user interface). At block 420, the process 400 receives tactile input from the operator and converts the input into one or more input signals. At block 430, the process 400 transmits the input signals to the operating system running, for example, on processor 250 and memory 260 (FIG. 2) and interprets the input signals as one or more recognized gestures. At block 440, an ultrasound application (stored, for example, in memory 260) receives the recognized gestures and provides corresponding instructions for an ultrasound engine configured to form ultrasound images. At block 450, the ultrasound engine generates one or more updated images based on the interpreted input from the operator. At block 460, the process 400 updates the user interface and the one or more generated ultrasound images are sent to the display.

### CONCLUSION

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof means any connection or coupling, either direct or indirect, between two or more elements; the coupling or connection between the elements can be physical, logical, or a combination thereof. Additionally, the words “herein,” “above,” “below,” and words of similar import, when used in this application, refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above Detailed Description of examples of the disclosed technology is not intended to be exhaustive or to limit the disclosed technology to the precise form disclosed above. While specific examples for the disclosed technology are described above for illustrative purposes, various equivalent modifications are possible within the scope of the disclosed technology, as those skilled in the relevant art will recognize. For example, while processes or blocks are presented in a given order, alternative implementations may perform routines having steps, or employ systems having blocks, in a different order, and some processes or blocks may be deleted, moved, added, subdivided, combined, and/or modified to provide alternative or subcombinations. Each of these processes or blocks may be implemented in a variety of different ways. Also, while processes or blocks are at times shown as being performed in series, these processes or blocks may instead be performed or implemented in parallel, or may be performed at different times. Further any specific numbers noted herein are only examples: alternative implementations may employ differing values or ranges.

The teachings of the disclosed technology provided herein can be applied to other systems, not necessarily the system described above. The elements and acts of the various examples described above can be combined to provide further implementations of the disclosed technology. Some alternative implementations of the disclosed technology may include not only additional elements to those implementations noted above, but also may include fewer elements.

These and other changes can be made to the disclosed technology in light of the above Detailed Description. While the above description describes certain examples of the disclosed technology, and describes the best mode contemplated, no matter how detailed the above appears in text, the disclosed technology can be practiced in many ways. Details of the system may vary considerably in its specific implementation, while still being encompassed by the disclosed technology disclosed herein. As noted above, particular terminology used when describing certain features or aspects of the disclosed technology should not be taken to imply that the terminology is being redefined herein to be restricted to any specific characteristics, features, or aspects of the disclosed technology with which that terminology is associated. In general, the terms used in the following claims should not be construed to limit the disclosed technology to the specific examples disclosed in the specification, unless the above Detailed Description section explicitly defines such terms.

I/We claim:

1. An ultrasound imaging system, comprising:
a display configured to present a user interface to an operator and to receive tactile input within the user interface from the operator; and
a processor, wherein the processor is configured to receive ultrasound measurements, wherein the processor is configured to generate one or more ultrasound images based on the ultrasound measurements, wherein the processor is configured to present the one or more ultrasound images at the display; wherein the processor is configured to generate one or more updated ultrasound images based on the received tactile input, and wherein the processor is configured to present the one or more updated ultrasound images at the display.

2. The system of claim 1, further comprising an input recognition engine configured to match the received tactile input to one or more associated actions, wherein the processor is configured to perform one or more associated actions to generate the updated ultrasound image.

3. The system of claim 2 wherein the user interface includes one or more connected lines overlaid onto an ultrasound image, wherein the one or more lines represent a boundary that surrounds an area of a region of interest in the ultrasound image, and wherein the processor is configured to recognize a touch input received along the boundary from at least one finger.

4. The system of claim 3 wherein the corresponding action comprises adjusting the size of the area bounded by boundary.

5. The system of claim 3 wherein the corresponding action comprises adjusting a shape of the area bounded by the boundary.

6. The system of claim 3 wherein the corresponding action comprises adjusting the position of the area bounded by the boundary.

7. The system of claim 3 wherein the processor is configured to recognize a touch input from at least one finger maintained in contact with the display, and wherein the corresponding action comprises adjusting the position of the area bounded by the boundary without changing the size and the shape bounded by the boundary.

8. The system of claim 2 wherein the processor is configured to recognize two successive taps received from at least two fingers, and wherein the corresponding action comprises freezing a output of the ultrasound image thereby presenting a static ultrasound image on the display.

9. The system of claim 2 wherein the processor is configured to recognize a touch input from a first finger and a second finger, wherein the first finger is separated from the second finger by a distance within the user interface, wherein the tactile input further comprises touch input received from the first finger rotating relative to the second finger while the distance between the first finger and the second finger within the user interface remains generally constant, wherein the ultrasound image is a Doppler image, and wherein the corresponding action comprises adjustment of an angle correction display in the Doppler image.

10. The system of claim 2 wherein the processor is configured to recognize a touch input from a first finger at a first location within the user interface corresponding to a first measurement point in the ultrasound image, wherein the corresponding action comprises presenting information on the display related to the first measurement point.

11. The system of claim 10 wherein the processor is further configured to recognize touch input from a second finger at a second location within the user interface corresponding to a second measurement point in the ultrasound image, wherein the corresponding action comprises presenting information on the display associated with a portion of the ultrasound image between the first and the second measurement points.

12. The system of claim 1 wherein the display comprises a touchscreen with a flat, cleanable surface.

13. The system of claim 1 wherein the display is a first display, and further comprising a second display larger than the first display.

14. A method of operating an ultrasound imaging system, the method comprising:
presenting a user interface at a touchscreen display, wherein the user interface includes a ultrasound image;
detecting a first tactile input at the display, wherein the first tactile input includes one or more input features;
converting the detecting tactile input to input signals;
matching the converted input signals to one or more associated actions;
generating an updated ultrasound image based on the associated action; and
presenting the updated ultrasound image within the user interface at the display.

15. The method of claim 14 wherein presenting the user interface includes overlaying one or more connected lines onto an ultrasound image, wherein the one or more lines represent a boundary that surrounds an area of a region of interest in the ultrasound image, and wherein detecting the first tactile input at the display comprises detecting a touch input received along the boundary from at least one finger maintained in contact with the display with the display for a predetermined amount of time.

16. The method of claim 15 wherein generating the updated ultrasound image comprises adjusting the position of the area bounded by the boundary without changing the size and the shape bounded by the boundary.

17. The method of claim 14,
wherein detecting the first tactile input at the display comprises detecting touch input from a first finger and a second finger;
wherein the first finger is separated from the second finger by a distance within the user interface;
wherein detecting the first tactile input at the display further comprises the detecting touch input received from the first finger rotating relative to the second finger while the distance between the first finger and the second finger within the user interface remains generally constant;
wherein the ultrasound image is a Doppler image; and
wherein generating an updated ultrasound image comprises adjusting a steering angle display in the Doppler image.

18. At least one computer-readable storage medium storing instructions for a method performed by an ultrasound device having a processor and a memory, the method comprising:
presenting a user interface at a touchscreen display, wherein the user interface includes a ultrasound image;
detecting a first tactile input at the display, wherein the first tactile input includes one or more input features;
converting the detected input features to input signals;
matching the converted input signals to one or more associated actions;
generating an updated ultrasound image based on the one or more associated actions; and
presenting the updated ultrasound image within the user interface at the display.
